(19) United States
(12) Patent Application Publication
(10) Pub. No.: US 2023/0321344 A1
(43) Pub. Date: Oct. 12, 2023

(54) SHROUD DEVICE

(71) Applicant: ELANCO US INC., Greenfield, IN (US)

(72) Inventors: Matthew Edward Andresen, Indianapolis, IN (US); Michael Roger, New Palestine, IN (US); Tony Jefferies, Greenfield, IN (US); David Andrew Trow, Hamilton (NZ); Brendan Lance Udy, Lincoln (NZ); David Bain Calder, Lugano (CH)

(21) Appl. No.: 18/207,959

(22) Filed: Jun. 9, 2023

Related U.S. Application Data
(63) Continuation of application No. PCT/US2021/062587, filed on Dec. 9, 2021.
(60) Provisional application No. 63/123,899, filed on Dec. 10, 2020.

(51) Int. Cl. A61M 5/162

(52) U.S. Cl. CPC A61M 5/162; A61M 2207/10; A61M 2250/00

(57) ABSTRACT

The disclosed embodiments include a protective housing for a fragile article for containing fluid medicament. The protective housing includes a shroud top including an upper collar for absorbing one or more forces, a shroud base configured to receive the shroud top and including a lower collar for absorbing one or more forces, a spike configured to pierce the fragile article and coupled to the shroud top or the shroud base, and a connector located at the shroud top or the shroud base, the connector configured to provide the fluid medicament from the fragile article to an injector

### FIELD

The present disclosure relates to a container for housing a medication contained within a fragile receptacle.

### BACKGROUND

A fluid medicament may be housed in a receptacle. The receptacle may be a bottle, vial, cartridge, or ampoule. The receptacle may include a pierceable closure such as a rubber stopper. Such receptacles may be made from medical grade glass which is not reactive with the medicament. Glass may break if dropped. This leads to loss of the medicament. Moreover, the spillage of the medicament may be a safety and health hazard to the user, environment or others in the vicinity.

To address this problem, the receptacle may be housed in a protective container. The protective container may be constructed as an applicator through which a user holds the receptacle and dispenses the medicament, e.g. by providing the container with an aperture through which a syringe needle can be inserted through the rubber stopper of the receptacle for withdrawal of a dose of the medicament into the syringe. While the receptacle may be designed to prevent bottle breakage and to assist usage in the field, other risks may persist. For example, the risk of accidental injection may be present in certain situations. The present disclosure presents embodiments to further reduce risks.

### SUMMARY

One implementation of the present disclosure is an apparatus including a protective housing for a fragile article for containing fluid medicament. The protective housing may include a shroud top including an upper collar for absorbing one or more forces, a shroud base configured to receive the shroud top and including a lower collar for absorbing one or more forces, a spike configured to pierce the fragile article and coupled to the shroud top or the shroud base, and a connector located at the shroud top or the shroud base, the connector configured to provide the fluid medicament from the fragile article to an injector. In one example, the fragile article comprises a glass bottle or other vessel for containing the fluid medicament. In one example, the protective housing includes a first indicium on the shroud top and a second indicium on the shroud base, wherein the first indicium aligns with the second indicium when the shroud base receives the shroud top. In one example, the spike is movable toward the fragile article through rotation of the shroud top with respect to the shroud base. The protective housing may include at least one inclined guide track coupled to the shroud top or the shroud base, the inclined guide track configured to move the shroud top toward the shroud base through rotation of the shroud top with respect to the shroud base. In one example, the at least one inclined guide track provides a path for the shroud top or the shroud based to push the spike through a seal in the fragile article. The protective housing may include a lock that secures the shroud base to the shroud top in a final position after the spike has pierced the fragile article. In one example, the protective housing may include a spike assembly coupled to the shroud top, the spike assembly comprising the spike. In one example, the spike assembly further comprises a cap configured to cover the connector and a tension clip configured to couple the spike assembly to the shroud top.

One implementation of the present disclosure is a method for using a protective housing including a shroud top and a shroud base configured to protect a sealed vial by absorbing one or more forces. The method comprises removing at least one tamper prevention device from the protective housing, rotating the shroud top with respect to the shroud base to pierce the sealed vial, and connecting an injector to the protective housing to provide a path for fluid medicament from the sealed vial after the sealed vial has been pierced. The method may include coupling a draw-off tube to the injector. The method may include coupling the draw-off tube to connector of the shroud top and the shroud base. The method may include priming the injector to ready fluid medicament to be injected to a subject.

One implementation of the present disclosure is a method for manufacturing a protective housing. The method includes one or more of providing a sealed vial containing fluid medicament to a shroud base including a lower collar for absorbing one or more forces on the protective housing, aligning a shroud top with the shroud base, shroud top including an upper collar for absorbing one or more forces on the protective housing, and connecting the shroud top and the shroud base to position a spike in a predetermined position such that the spike configured to pierce the sealed vial. The method may include coupling a connector to the shroud top or the shroud base, the connector configured to provide fluid medicament from the sealed vial to an injector. The method may include aligning a first indicium on the shroud top with a second indicium on the shroud base. In one example, the spike is movable toward the sealed vial through rotation of the shroud top with respect to the shroud base. The method may include providing a lock on the shroud base to the shroud top that secures the shroud base to the shroud top in a final position after the spike has pierced the fragile article. The method may include coupling a spike assembly to the shroud top, the spike assembly comprising the spike. The method may include applying a press jig to the shroud top or the shroud base to snap the shroud top and shroud base together.

### BRIEF DESCRIPTION OF THE DRAWINGS

Exemplary embodiments are described herein with reference to the following drawings, according to an exemplary embodiment.

FIG. 1 illustrates an example protective shroud to house and dispense medicament.

FIG. 2 illustrates an exploded view of the shroud of the container system.

FIG. 3 illustrate an exploded view of the spike assembly for the shroud.

FIG. 4 illustrates an example injector compatible with the protective shroud.

FIG. 5 illustrates an example flowchart for the manufacturing of the protective shroud.

FIG. 6 illustrates a sealed vial enclosed by the protective shroud.

FIG. 7 illustrates an alignment of the top and base of the protective shroud.

FIG. 8 illustrates the top and base of the protective shroud press fit together.

FIG. 9 illustrates an example flow chart for the use of the protective shroud.

FIG. 10 illustrates a removal of tamper prevention device.

FIG. 11 illustrates rotation of the top with respect to the base of the protective shroud.

FIG. 12 illustrates removal of a cap of the protective shroud.

FIG. 13 illustrates the connection tube coupled to the protective shroud.

FIG. 14 illustrates dispensing of the fluid medicament.

### DETAILED DESCRIPTION

The following embodiments include improvements in a receptacle and dispenser for a medicament. The medicament may be a fluid, a gel, a paste or another material. A variety of medicaments may be used. The medicament may be a bovine respiratory disease antibiotic. The medicament may be provided in a glass bottle, a plastic container, vial, cartridge, ampoule, or another type of vessel. The following description may refer to a “bottle”, but it is understood that any of these vessels may be substituted. A protective container is particularly needed in the instance of a glass bottle because the glass bottle may be used in conditions where there is a likelihood of it being dropped and broken, especially when trying to insert a needle into the stopper of the bottle, e.g. because an animal is stressed and therefore difficult to handle, the medicament is being administered outdoors in adverse weather conditions and/or the medicament is being administered while riding horseback or an all-terrain vehicle. Further, it is not always easy to hold the protective container and to insert a syringe needle through the container aperture into the stopper.

In some examples, a dose of the fluid medicament from a vessel housed in a protective container may be withdrawn by inserting a needle of a syringe through a closure or seal (e.g., rubber stopper) via an aperture. With a single use needle this is an effective and efficient technique for removing the medicament from the vessel and administering the medicament to a subject, one or more challenges may arise from this technique. For example, when a user both holds the receptacle and dispenses the medicament using the syringe needle, there may be a risk of accidental exposure of the medicament to the user. The user may be accidentally injected with the medicament, or the medicament may be accidentally spilled onto the user.

In addition, an alternative technique for discharging a dose of the fluid medicament from the receptacle housed in the protective container may be used. For example, a nozzle accessory for discharging the fluid medicament may be maneuvered to pierce the closure to provide a discharge path for the fluid medicament.

However, a user may still opt to insert a needle of a syringe to extract the fluid medicament, introducing the same challenges of a risk of accidental exposure or spillage. That is, the user may choose not to follow the recommend procedure for administering the fluid medicament, which causes a risk of exposure or spillage. The following embodiments describe a shroud that prevents access via a single user needle. The structure protects the user and improves the overall safety of the device and product handling. The shroud is configured for a user to cause insertion of an internal spike to penetrate the stopper to allow the medicament to be dispensed using a multi-use injector device. The spike may be formed of plastic, metal, or another material with a stiffness sufficient to penetrate the stopper. The spike is tubular, having an internal pathway. The spike is maneuvered so that the sharp distal tip of the spike pierces the stopper to provide a discharge path for the fluid medicament through the spike.

Moreover, at least one of the following embodiments prevent the user from access to the fluid medicament through multiple redundant safety measures. In addition to the overall structure that encases the receptacle of the fluid medicament, a second safety measure is a tamper prevention device that prevents the user from opening the device. Finally, a third safety measure includes one or more valves that prevent backflow with the fluid medicament is dispensed.

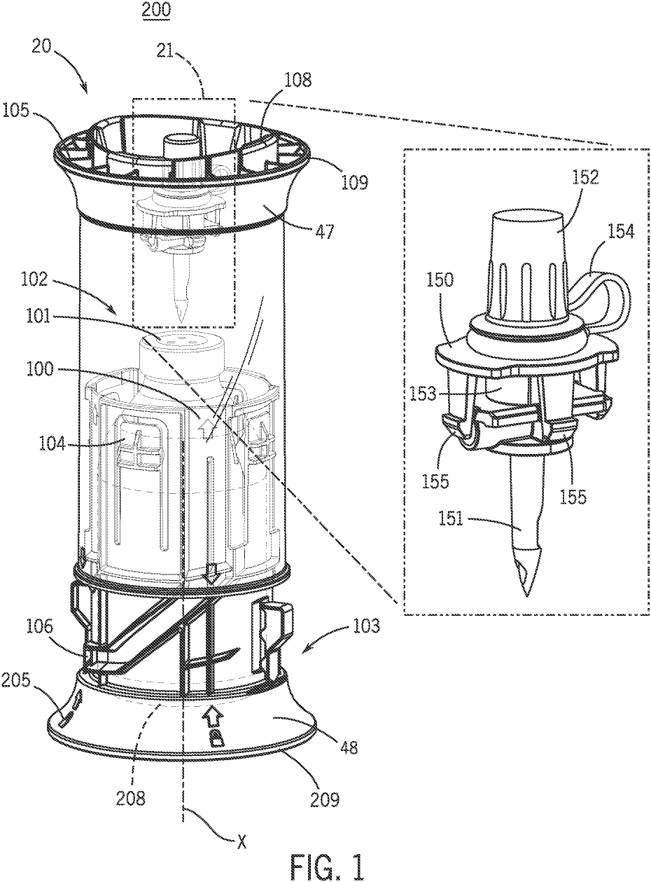

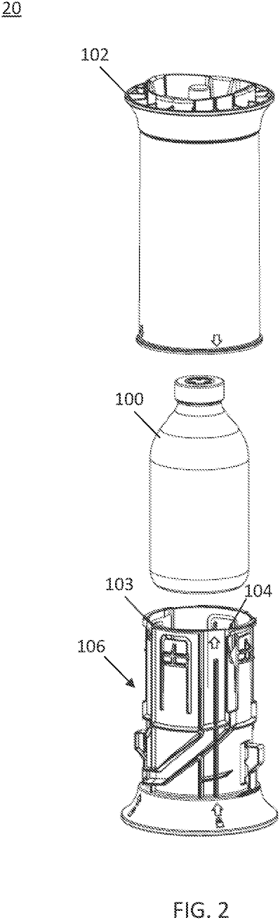

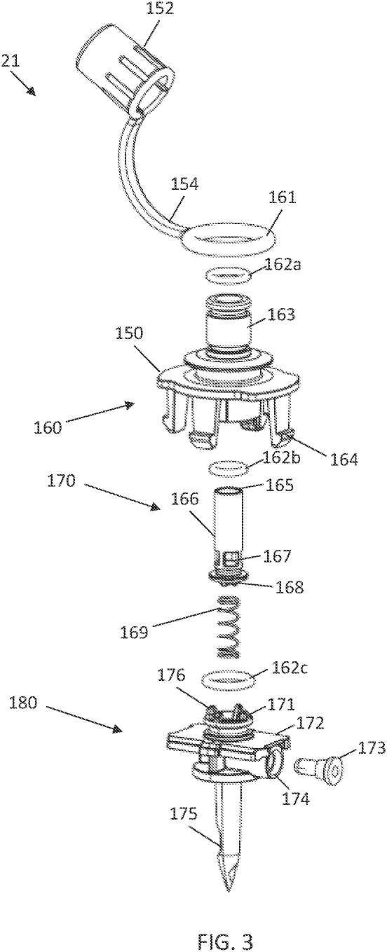

FIG. 1 illustrates an example container system including a protective container assembly or shroud 20 to dispense medicament contained in a sealed vial or fragile container 100, which in example type of fragile article and may be a bottle. The shroud 20 may include a spike assembly 21 for puncturing and obtaining the medicament from the fragile container, an upper collar 47, a lower collar 48, and a cylindrical container between the upper collar 47 and the lower collar 48 that provides the housing for the fragile container 100. The fragile container 100 can be any size such as 50 mL, 100 mL, 250 mL, 500 mL, and 1000 mL. The following structure may be adapted to any size. Additional, different, or fewer components may be included. FIG. 2 illustrates an exploded view of the shroud 20 of the container system. Details of the spike assembly 21 are described in reference to FIG. 3.

The container is of a generally cylindrical form with a longitudinal axis X. The shroud top 102 may be defined by an outer wall structure having a predetermined thickness. Generally, the outer wall structure has a thickness in the range of approximately 1.0-2.0 mm, preferably in the range of 1.3-1.7 mm. Preferably, the protective shroud top 102 is made from polypropylene by injection molding and has a length of approximately 1-40 cm. Polypropylene may have one or more properties that cause the material be to deflectable and absorb impact forces. Other materials capable of absorbing forces may be included. Other materials may be used instead of polypropylene such as acrylonitrile butadiene styrene, polyamide, glass filled polyamide, resins, and polycarbonate. Besides injection molding 3D printing or laser sintering, or other manufacturing processes may be used.

The upper collar 47 absorbs one or more forces placed on the shroud 20 in order to protect the fragile container 100. The upper collar 47 includes an inner rim 108 and an outer rim 109, which may be connected by one or more ribs 105 that extend between the inner rim 108 and the outer rim 109 in a direction perpendicular to the inner rim 108 and/or the outer rim 109. In other words, the ribs 105 are vertically shaped and positioned radially from the outer circumference of the upper collar 47 toward the center of the upper collar 47. The length of the ribs 105 may extend to the spike assembly 21.

The lower collar 48 is coupled to the shroud base 103 and configured to receive and absorb one or more forces. The lower collar 48 includes an inner rim 208 and an outer rim 209, which may be connected by one or more ribs 205 that extend between the inner rim 208 and the outer rim 209 in a direction perpendicular to the inner rim 208 and/or the outer rim 209.

In both the lower collar 48 and the upper collar 47 are associated with ribs. The ribs may be adjacent to, or coupled with, the corresponding inner rim and outer rim to form hollow spaces that are semi-enclosed. The hollow spaces may be shaped as an annulus. The inner radius of the annulus is defined by the radius of the inner rim and the outer radius of the annulus is defined by the radius of the outer rim. The distance (e.g., angular distance) between ribs defines the angular span or size of the hollow spaces.

The size of the hollow spaces, the material of the lower collar 48 or the upper collar 47, the size of the lower collar 48 or the upper collar 47, or other features to absorb forces may be referred to as the “configurable shock absorbing features” of the shroud 20. The configurable shock absorbing features may be selected based on the size or mass of the shroud 20, for example including the vessel and medicament. The configurable shock absorbing features may be adjusted according to the type of medicament, the volume of medicament, or another consumable substance. The configurable shock absorbing features may be selected according to the intended recipient of the medicament (e.g., type of animal). That is the size of the hollow spaces may or the dimensions of the collars may be selected to a first value for a first animal type and selected as a second value for a selected animal type.

Either or both of the lower collar 48 and the upper collar 47 may be removable from the protective container of the shroud 20. For example, the lower collar 48 and/or the upper collar 47 may include a protrusion on the inside surface that snap fits into an indention on the protective container. In another example, the lower collar 48 and/or the upper collar 47 may screw into the protective container of the shroud 20. In another embodiment, the lower collar 48 and the upper collar 47 may be fixedly coupled to the shroud 20. The lower collar 48 and the upper collar 47 may be integrally formed with the protective container.

If the shroud 20 is dropped on either the lower collar 48 or the upper collar 47, the shroud 20 will land on the raised perimeter of the hollow spaces. Because the upper collar 47 and the lower collar 48 have a diameter and/or other dimension that is greater than the cylindrical container, the shroud always lands on the upper collar 47 and/or the lower collar 48. The lower collar 48 or the upper collar 47 deflects towards the shroud 20 and radially outwardly/inwardly to absorb the impact forces. Thus, the likelihood of breakage of the vessel 100 is reduced.

As shown in FIG. 2, the shroud top 102 fits over the shroud base 103 to enclose the vessel 100. The shroud base 103 is cylindrical and includes one or more features that interact with the vessel 100 and one or more features that interact with the shroud base 103. Internal features or cushion members 104 are on the inside of the shroud base 103 and take many forms. External features such as guide members 106 are on the outside of the shroud base 103 and also take many forms.

The features that interact with the vessel 100 may include one or more spacing or cushion members 104 may include a series of panels and/or ridges. The spacing or cushion members 104 provide a space around the vessel 100. The spacing or cushion members 104 provide impact resistance and absorb side-on impact energy by deflecting into the space created between them and the vessel 100.

The features that interact with the shroud base 103 may include one or more guide members 106 may provide a track or guide for the shroud top 102 to rotate with respect to the shroud base 103 into a seated position. For example, the guide members 106 may move the relative position of the shroud top 102 and the shroud base 103

A locking mechanisms for releasably locking the shroud top 102 and the shroud base 103 may be used. As an example, other forms of snap fits, for instance based on inter-engaging complementary surfaces, such as stepped surfaces, may be include on the outer surface of the shroud base 103 and/or the inner surface of the shroud top 102. The locking mechanism may also be actuated by movement of the shroud base 103 relative to the shroud top 102 other than by rotation or screwing.

As discussed in more detail below, the shroud top 102 and/or the shroud base 103 may include one or more indicia 131 to aid the user, a technician, or an automated machine in assembling the shroud 20. In one example, the shroud 20 includes a first indicium on the shroud top 102 and a second indicium 131 on the shroud base. Alignment of the first indicium aligns with the second indicium when the shroud base receives the shroud top.

FIG. 3 illustrates an exploded view of the spike assembly 21 for the shroud. The spike assembly 21 includes an injector mating portion 160, a valve portion 170 and a vessel piercing portion 180. The injector mating portion 160 includes is a connector located at the shroud top 102, as illustrated, but could be coupled to the shroud base 103 in another embodiment. The injector mating portion 160 is configured to provide the fluid medicament from the fragile article to an injector.

The injector mating portion 160 includes a cap 152, a tether 154, a tether ring 161, one or more seals (e.g., connector o-ring 162a), a collar 163, a locking plate 150, and one or more locking arms 164. The tether ring 161 may connect the tether 154 and cap 152 to the locking plate 150, which is secured directly to the valve portion 170 and indirectly to vessel piercing portion 180. The connector o-ring 162a may be configured to fit around collar 163. The connector o-ring 162a may create a seal with the cap 152 to create a tight fit between the cap 152 and the collar 163. The cap 152 is configured to cover the connector and a tension clip configured to couple the spike assembly to the shroud top.

The locking plate 150 may include any number of locking arms 164. The example illustrated includes four locking arms 164. The locking arms 164 may include a flexible arm with a tab on the end of the flexible arm. When the locking plate 150 is pushed into the shroud top 102 the flexible arms are pushed inward and the tabs lock the locking plate 150 and the injector mating portion 160 to the shroud top 102. Alternatively, the locking plate 150 may be mated with the upper collar 47 such that the flexible arms are pushed inward, and the tabs lock the locking plate 150 and the injector mating portion 160 to the shroud top 102.

The valve portion 170 includes one or more seals (e.g., valve o-ring 162b), a valve 166, and a valve spring 169. The valve portion 170 regulates the flow of medicament from the shroud top 102 to the spike assembly 21. The valve 166 includes one or more windows 167 that supply medicament through the valve 166 to a downstream opening 165. Four windows 167 are illustrated in valve 166.

The valve 166 includes a spring lip 168. The spring lip 168 secured the valve spring 169 to the valve 166. The valve spring 169 biases the valve 166 to a closed position so that medicament does not flow through the valve 166. In one example, the valve spring 169 pushes the valve 166 into collar 163 so that windows 167 are blocked by the collar 163. When the spike assembly 21 is pressed down to the vessel 100 by the shroud top 102 as it fits over the shroud base 103. Thus, the spike assembly 21 is movable toward the fragile article through rotation of the shroud top 102 with respect to the shroud base.

The vessel piercing portion 180 includes one or more seals (e.g., spike o-ring 162c), a fastener 176, and a chamber 171, a spike platform 172, a split valve 173, a passageway 174, and a piercing point or stem 175. The spike o-ring 162c may provide a seal between the vessel piercing portion 180 and the valve portion 170. The chamber 171 provides a flow path for the medicament to flow from the piercing point 175 to the valve portion 170 and the injector mating portion 160. The piercing point 175 may be referred to as a spike.

The split valve 173, or release valve, includes a shaft that seats within the passageway 174. A split in the shaft is configured to open or close under pressure. A vacuum may build up in the vessel 100 during dispensing as the contents are discharged through the nozzle. The passageway 174 with the split valve 173 provides a vent for the vessel 100 allowing easy evacuation of the product from the vessel 100 and avoids a vacuum build-up which makes injection inaccurate, slow and difficult. This would also be the case for other rigid containers. Such a vent system, however, may be omitted when the vessel 100 were of a collapsible type. In one example, one or more locking arms 164 covers the split valve 173 to mechanically hold the split valve 173 within the passageway 174.

The piercing stem 175 may include multiple passageways. A main passageway may extend from the opening in the tip of the piercing point 175 through the longitudinal axis to the injector mating portion 160 and/or valve portion 170. A second passageway extends in a perpendicular axis (e.g., perpendicular to the longitudinal axis) to the passageway 174 with the split valve 173 in order to release pressure from the vessel during dispensing the medicament. That is the second passageway vents the vessel 100. Also shown in FIG. 3, the piercing stem 175 tapers inwardly and the tip of the piercing stem 175 has a notch to provide a sharpened point for piercing the closure of the vessel 100.

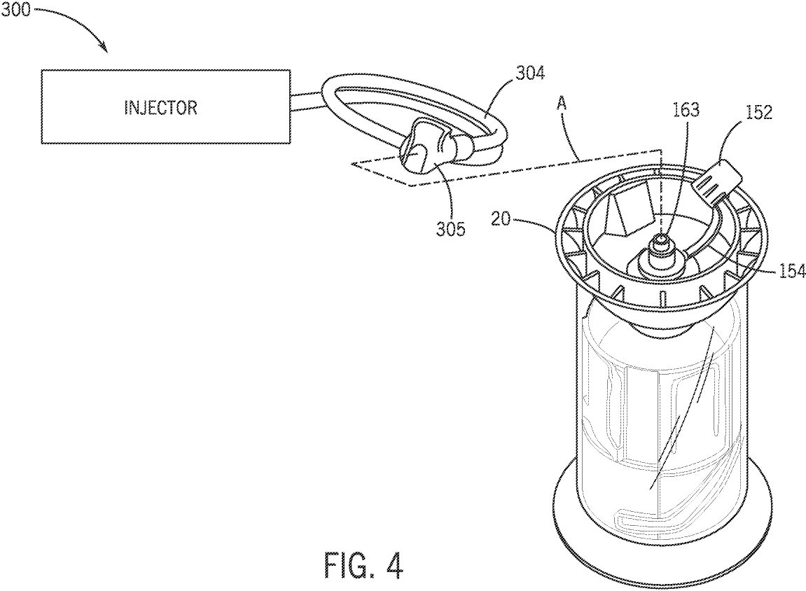

FIG. 4 illustrates an example injector 300 compatible with the shroud 20. The injector 300 may include a handle, a trigger, a needle, and a shield. The injector 300 is connected to the shroud 20 through a tube 304 and connector 305. The user opens the cap 152 to reveal the collar 163. The connector 305 is pushed onto the collar 163, as shown by arrow A. The valve portion 170 is opened and allows fluid to flow only when the valve portion 170 is depressed by fitting the connector 305. The user grips the handle and pulls the trigger, which actuates a pump inside the injector 300 to draw the fluid medicament from the vessel 100, through the tube 304, through the injector 300 and out the needle that extends through the shield. Other types of injectors may be used.

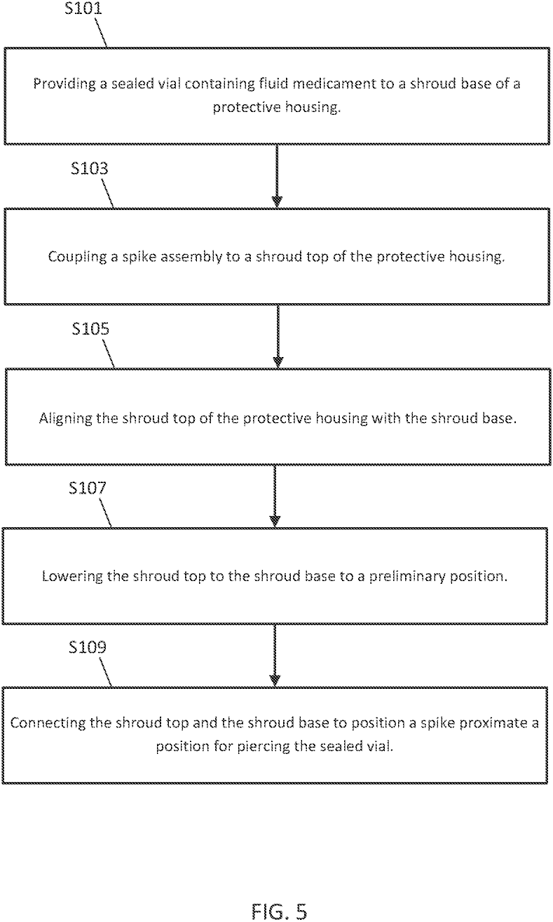

FIG. 5 illustrates an example flowchart for the manufacturing of the protective shroud. The actions of the flowchart may be performed by a user (e.g., medical professional, animal health professional, or a manufacturer) or they may be performed by an assembly system. The assembly system may include one or robotic arms or otherwise automated devices for assembly of the protective shroud. The robotic arms or automated devices may receive instructions from a controller to drive a motor, actuator, or another device to facilitate the actions of FIG. 5 for manufacturing of the protective shroud. The controller may include a memory for storing instructions for generating commands for the motor, actuator, or other device, and the controller may include a processor for performing the instructions, generating the commands, and sending the commands to the motor, actuator, or other device. Additional, different, or fewer components and/or actions may be included.

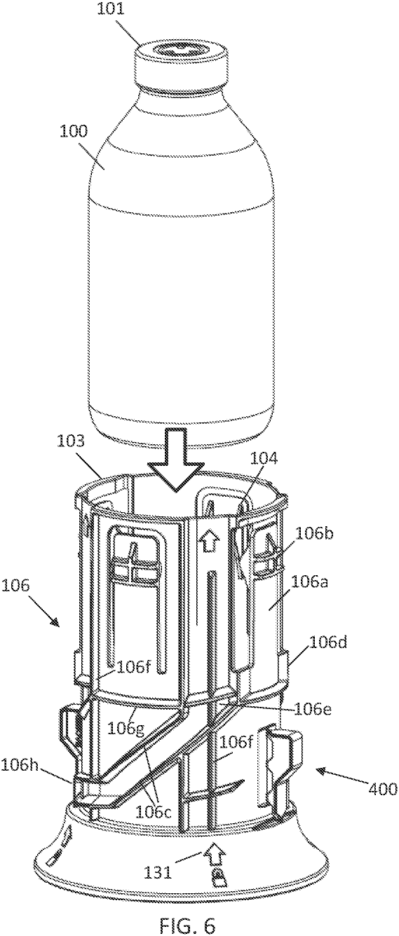

Initially, in action S101, a sealed vial (e.g., vessel 100) is provided. The sealed vial contains fluid medicament to the shroud base 103 including a lower collar 48 for absorbing one or more forces on the protective housing. The sealed vial may include a closure 101. The sealed vial may be provided to a user or at a manufacturing location. The manufacturing process may include manually providing the sealed vial to the shroud base 103 by lowering the sealed vial into the shroud base 103 (e.g., as illustrated in FIG. 6). The manufacturing process may include automatically providing the sealed vial to the shroud base 103 using a mechanical arm under the command of the controller.

In action S103, the spike assembly 21 is coupled to the shroud top 102 of the protective housing. The spike assembly 21 may snap into the shroud top 102. The manufacturing process may include manually coupling the spike assembly 21 to the shroud top 102. The manufacturing process may include automatically pressing the spike assembly 21 into the shroud top 102 using a jig under the command of the controller.

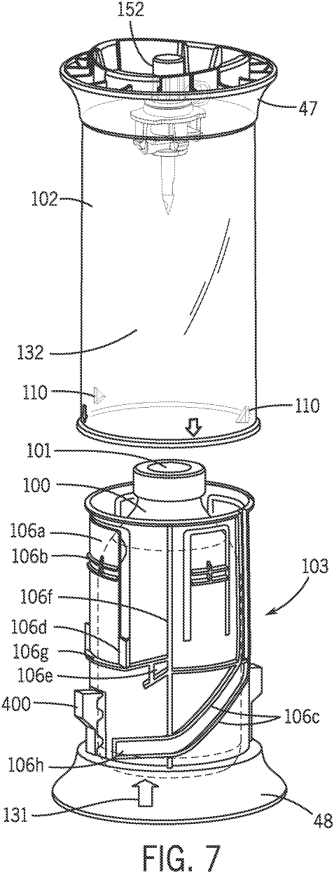

In action S105, the shroud top 102 of the protective housing is aligned with the shroud base 103. In action S107, the shroud top 102 is lowered to the shroud base 103 to a predetermined position. FIG. 7 illustrates the sealed vial enclosed by the protective shroud 20 as the shroud top 102 is lowered onto the shroud base 103. Certain features of the shroud base 103 and/or shroud top 102 may be better visible in FIG. 6. The guide members 106 of the shroud base 103 may include a finger 106a, a tab ridge 106b, circumferential ridges 106c and 106g, a shroud spacer 106d, security alignment recess 106e, a longitudinal ridge 106f, and a lock 106h.

The fingers 106a may function as biased tabs to grip the vessel 100 when it is inside or mated with the shroud base 103. The finger 106a has one or more physical properties (e.g., coefficient of elasticity) that cause the finger 106a to exert a force (e.g., holding force) on the vessel 100. The fingers 106a have inwards-facing ribs that locate over the shoulder of the vial to prevent the vial moving in an axial direction. On the interior of the shroud base 103, the finger 106a may include a high friction surface that is scored, includes a surface feature adhered with adhesive, or otherwise creates a grip member to increase the friction between the shroud base and the vessel 100.

In addition, or in the alternative, the tab ridge 106b applies a force to the finger 106a to bias the tab toward the vessel 100. The tab ridge 106b has a width such that an outer edge of the ridge comes in contact with the shroud top 102 when the shroud top 102 is placed on the shroud base 103. Thus, the shroud top 102 pushes the finger 106a toward the vessel 100 to grip the vessel 100 when the shroud top 102 mates with the shroud base 103. The shroud base 103 also includes one or more shroud spacers 106d that regulate the spacing between the shroud top 102 and the shroud base 103. The tab ridge 106b may include a width that is greater than the width of the shroud spacers 106d. Thus, the tab ridge 106b applies the force to the finger 106a for a predetermined radial length limited by the width of the shroud spacers 106d. The predetermined radial length may be equal to a difference between the shroud spacers 106d and the tab ridge 106b.

The security alignment recess 106e may be included in any quantity on the shroud base 103. The security alignment recess 106e aligns the shroud top 102 to the shroud base 103 at the position that allows the shroud top 102 to be properly seated to the shroud base 103. The tamper resistance system may be dependent on the security alignment recess 106e. Opposing the security alignment recess 106e, on the shroud top 102, is a protrusion or tab (e.g., security alignment protrusion 110 on the shroud top 102) that fits inside the security alignment recess 106e. The only radial alignment between the shroud base 103 and the shroud base 103 that results in a tight fit (e.g., a snap fit) is when the security alignment recess 106e aligns and snaps together with the protrusion or tab of the shroud top 102. The indicia 131 may include an arrow and or a lock to aid in alignment of the security alignment recess 106e with the shroud top 102.

As illustrated by FIGS. 6 and 7, a pair of the slanted circumferential ridges 106c form an inclined guide track coupled shroud base 103. The inclined guide track configured to move the shroud top toward the shroud base 103 through rotation of the shroud top 102 with respect to the shroud base 103. The inclined guide track may alternatively be coupled to the to the shroud top 102. The at least one inclined guide track provides a path for the shroud top 102 or the shroud base 103 to push the spike through a seal in the vessel 100.

At the end of the inclined guide track is a lock 106h that secures the shroud base 103 to the shroud top 102 in a final position after the spike has pierced the vessel 100. The lock 106h may be a recess on the shroud base 103 that mates with a protrusion or tab on the shroud top 102. Alternatively, the lock 106h may include a protrusion or tab that mates with a recess or other mechanism on the shroud top 102. In either example, the lock 106h may include a one-way locking mechanism such that once the shroud top 102 has been locked with the shroud base 103, it cannot be reverse or removed with destroying the shroud 20. The lock 106h may include a self-engaging prong that connects to the shroud base 102 as is not reversible. As discussed above, the indicia 131 may include an arrow to aid in alignment of the shroud top 102 and shroud base 103, but also may be included an indicator (e.g., a lock symbol) to reference the radial position where corresponding indicia on the shroud top 102 should line up when the lock 106h has been engaged. The lock 106h on the shroud base 103 secures the shroud base 103 to the shroud top 102 in a final position after the spike has pierced the fragile article.

The security alignment recess 106e may be any shape. As illustrated, the security alignment recess 106e may be a triangular prism or a triangle. Other shapes include circular, spherical, half spherical, or a rectangular prism. The hypotenuse of the triangle or cross section of the triangular prism is parallel and co-linear with at least one slanted circumferential ridge 106c of the shroud base 103. A base or leg of the triangle may be parallel and co-linear with at least one longitudinal ridge 106f of the shroud base. The other base or leg of the triangle is parallel and co-linear with at least one horizontal circumferential ridge 106g.

In one example, the user aligns the shroud top 102 with the shroud base 103 using the indicia 131, which may include a first indicium on the shroud top 102 and a second indicium on the shroud base 103. At a subsequent time or simultaneously, the user lowers the shroud top 102 onto the shroud base 103. Thus, the manufacturing process may include manually aligning and lowering the shroud top 102 to the shroud base 103 through sight and the indicia 131. The manufacturing process may include automatically aligning and lowering the shroud top 102 to the shroud base 103 using a mechanical arm under the command of the controller and/or an optical sensor that reads and/or detects the indicia 131. In this example, the indicia 131 may include a line, code, cross hair, or other optically readable symbol. Also visible in FIGS. 6 and 7 is a tamper prevention device 400, which is described in more detail below. The tamper prevention device 400 includes one or more removable components that provides, under visual inspection, whether the shroud 20 has been tampered with.

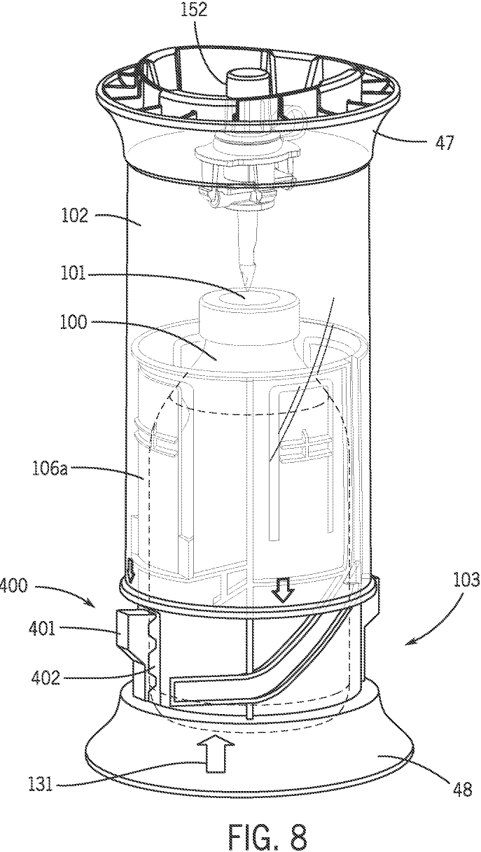

In action S109, the shroud top 102 and the shroud base 103 are connected to position the spike assembly 21 proximate to a position for piercing the sealed vial. FIG. 8 illustrates the shroud top 102 and the shroud base 103 of the protective shroud press fit together. The manufacturing process may include manually pressing the shroud top 102 into position so that the spike assembly 21 is in position just above the sealed vial. The manufacturing process may include automatically pressing the shroud top 102 over the shroud base 103 using a mechanical arm or a press jig under the command of the controller. The press jig may be configured to snap fit the shroud top 102 and the shroud base 103 together to fixedly coupled the shroud top 102 and the shroud base. As a result, the spike assembly 21 is movable toward the sealed vial through rotation of the shroud top 102 with respect to the shroud base 103. The position of the shroud top 102 may be above the tamper prevention device 400. The tamper prevention device 400 may include a removable tab 401 with thin connectors 403 that are bendable.

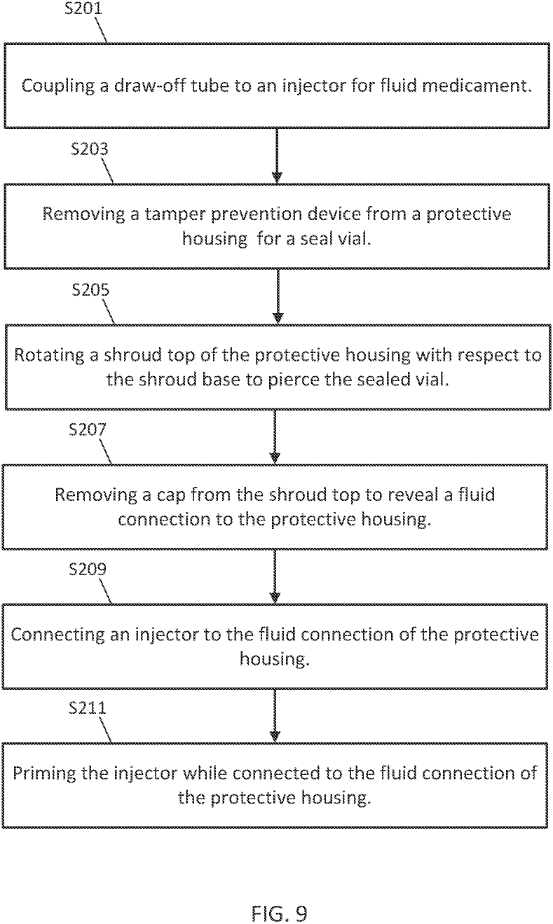

FIG. 9 illustrates an example flow chart for the use of the protective shroud. The actions of the flowchart may be performed by a user (e.g., medical professional, product producer or animal health professional) as the medicament is applied to a subject (e.g., animal). In other examples, the use of the protective shroud is automated using an administering system including a controller for one or more of the following actions for application of the protective shroud to deliver the medicament to the subject.

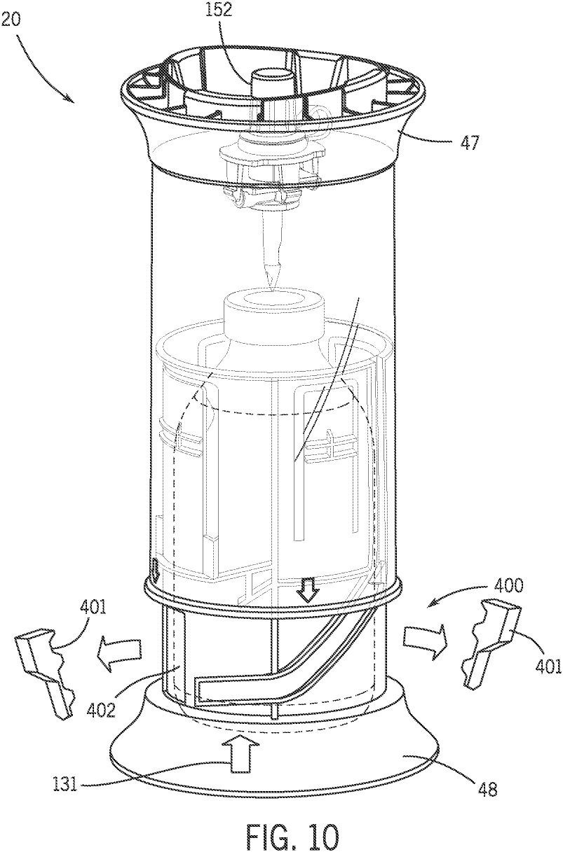

In action S201, a draw-off tube (e.g., tube 304) is coupled to the injector 300 for dispensing fluid medicament. In action S203, the tamper prevention device 400 is removed from the protective housing for the sealed vial (e.g., vessel 100). The user may visually inspect the shroud 200 to make sure the tamper prevention device 400 is present on all appropriate sides (any number of tamper prevention devices 400 may be used). To remove the tamper prevention device 400 the user may grip the removable tab 401 and apply a force in one or more direction (e.g., back and forth in a circumferential direction) to break the thin connectors 403. The connectors 403 may be formed of a different material than the removable tab 401 and/or formed of a different material than the rest of the shroud base 103. FIG. 10 illustrates a removal of tamper prevention device. Once the removable tab 401 is removed, the shroud top 102 is free to be moved using the inclined guide in a downward direction (e.g., in the direction of gravity).

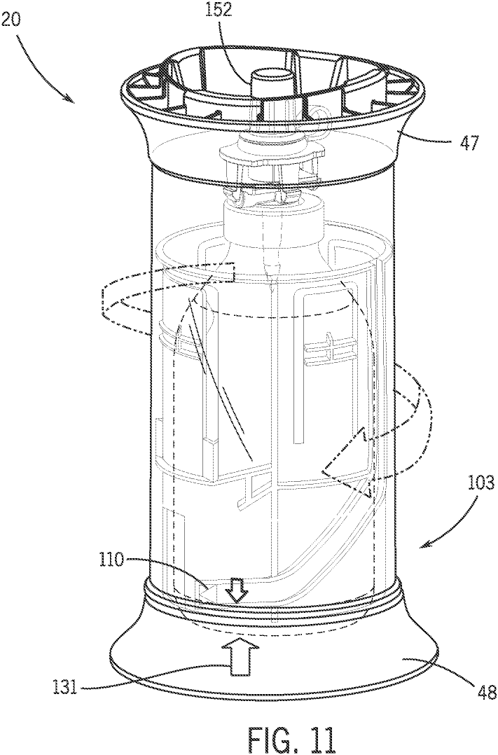

In action S205, the shroud top 102 of the protective housing is rotated with respect to the shroud base 103 to pierce the sealed vial. FIG. 11 illustrates rotation of the top with respect to the base of the protective shroud. The piercing stem 175 of the spike assembly 21 is pushed through the closure 101 of the sealed vial. The inclined guide track provides a path to guide the shroud top 102 in a predetermined amount of rotation or predetermined distance. The predetermined amount of rotation may be set according to the circumferential length of the inclined guide track. The predetermined amount of rotation may be ¼ turn or 90 degrees. Other angles are possible. In this example, in action S205 the shroud top 102 is rotated ¼ turn with respect to the shroud base 103 such that a protrusion 110, formed on the inner surface of the shroud top 10, travels from the security alignment recess 106e to the lock 106h. The protrusion 110 may be any shape (e.g., triangular, rectangular, circular, or three-dimensional projections thereof) and may be shaped to match the security alignment recess 106e so that the protrusion 110 fits with the security alignment recess 106e.

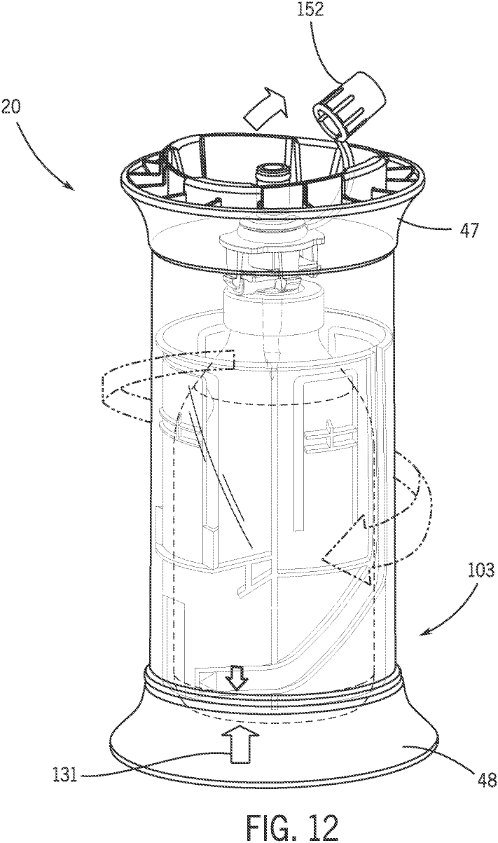

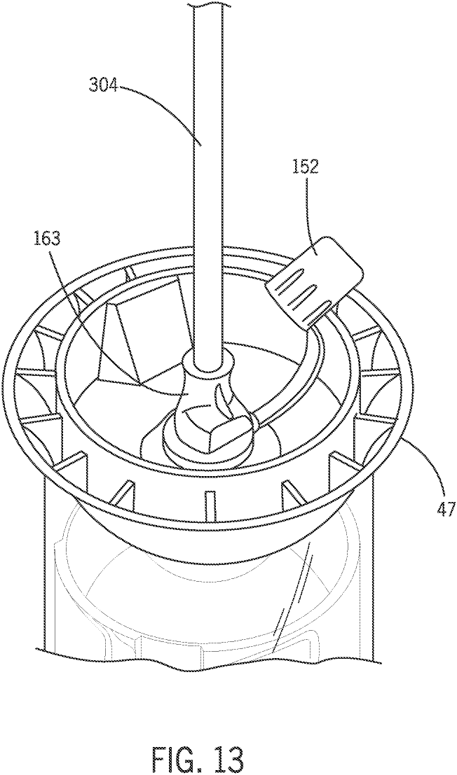

In action S207, the cap 152 is removed from the shroud top to reveal a fluid connection to the protective housing. FIG. 12 illustrates removal of the cap 152 of the protective shroud. The cap 152 may be removed manually by the user or using a tool (e.g., pliers). In action S209, the injector 300 is connected to the fluid connection of the collar 163 of the protective housing via the connection tube 304. FIG. 13 illustrates the connection tube 304 coupled to the shroud 20. The connection tube 304 may be pushed onto the collar 163 manually or using a tool (e.g., pliers).

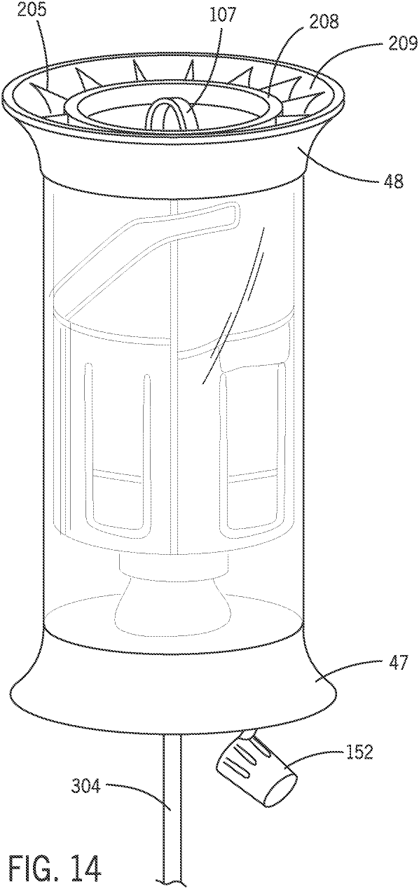

In action S211, the injector 300 is primed while connected to the fluid connection of the shroud 20. Priming the injector 300 may include pumping the handle and/or trigger one or more times to advance the medicament through the connection tube 304. FIG. 14 illustrates dispensing of the fluid medicament from the shroud 20. A fixed or static hanger 107 for hanging the protective container in an inverted position may be coupled to the lower collar 4. The user may invert the shroud 20 and hang the shroud on a fixed point or structure using the hanger 107. The shroud 20 may be hanged before priming, but also may be held or placed in any location or orientation. Once primed, the user may insert the needle into the subject and continue to pump the handle and/or the trigger to dispense the fluid medicament through the needle into the subject.

It will be understood by the skilled reader that the present invention is not limited to the exemplary embodiments herein described and that many variations and modifications may be made within the scope of the appended claims. For example, other flexible or resilient materials beside polypropylene (plastics and otherwise) may be used for the protective container. In addition, the collars of the protective container may be discontinuous, e.g. in the form of a broken ring of discrete beads. Alternately, the collars may be dispensed with and the panel spacing on the side of the container part selected such that the protective container will always land on one of the ribs in a side-on impact. Furthermore, the base part may be permanently connected to the container part and movable between open and closed positions to allow insertion and withdrawal of the vessel, e.g. through a hinged connection.

It will also be understood by the skilled reader that the present invention is not restricted to the provision of a protective container for a liquid medicament. The protective container of the invention has application for other receptacles and contents to be dispensed, whether in liquid form, powder form, or some other form, as well as application for storage and transportation of fragile articles in general, e.g. ceramic or glass structures.

If included, the controller may include a processor such as a specific purpose processor, an application specific integrated circuit (ASIC), one or more programmable logic controllers (PLCs), one or more field programmable gate arrays (FPGAs), a group of processing components, or other suitable processing components. The processor is configured to execute computer code or instructions stored in memory or received from other computer readable media (e.g., embedded flash memory, local hard disk storage, local ROM, network storage, a remote server, etc.). The processor may be a single device or combinations of devices, such as associated with a network, distributed processing, or cloud computing. If included, the memory may include one or more devices (e.g., memory units, memory devices, storage devices, etc.) for storing data and/or computer code for completing and/or facilitating the various processes described in the present disclosure. A computer-readable medium may include single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein. In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored. The computer-readable medium may be non-transitory, which includes all tangible computer-readable media.

What is claimed is:

1. A protective housing for a fragile article for containing fluid medicament, the protective housing comprising:
a shroud top including an upper collar for absorbing one or more forces;
a shroud base configured to receive the shroud top and including a lower collar for absorbing one or more forces;
a spike configured to pierce the fragile article and coupled to the shroud top or the shroud base; and
a connector located at the shroud top or the shroud base, the connector configured to provide the fluid medicament from the fragile article to an injector.

2. The protective housing of claim 1, wherein the fragile article comprises a glass bottle, a plastic container, or a cartridge for containing the fluid medicament.

3. The protective housing of claim 1, further comprising:
a first indicium on the shroud top; and
a second indicium on the shroud base, wherein the first indicium aligns with the second indicium when the shroud base receives the shroud top.

4. The protective housing of claim 1, wherein the spike is movable toward the fragile article through rotation of the shroud top with respect to the shroud base.

5. The protective housing of claim 1, further comprising:
at least one inclined guide track coupled to the shroud top or the shroud base, the at least one inclined guide track configured to move the shroud top toward the shroud base through rotation of the shroud top with respect to the shroud base.

6. The protective housing of claim 5, wherein the at least one inclined guide track provides a path for the shroud top or the shroud based to push the spike through a seal in the fragile article.

7. The protective housing of claim 1, further comprising:
a lock that secures the shroud base to the shroud top in a final position after the spike has pierced the fragile article.

8. The protective housing of claim 1, further comprising:
a spike assembly coupled to the shroud top, the spike assembly comprising the spike and at least one release valve to vent the fragile article.

9. The protective housing of claim 8, wherein the spike assembly further comprises a cap configured to cover the connector and a tension clip configured to couple the spike assembly to the shroud top.

10. A method for manufacturing a protective housing, the method comprising:
providing a sealed vial containing fluid medicament to a shroud base including a lower collar for absorbing one or more forces on the protective housing;
aligning a shroud top with the shroud base, shroud top including an upper collar for absorbing one or more forces on the protective housing; and
connecting the shroud top and the shroud base to position a spike in a predetermined position, the spike configured to pierce the sealed vial.

11. The method of claim 10, further comprising:
coupling a connector to the shroud top or the shroud base, the connector configured to provide fluid medicament from the sealed vial to an injector.

12. The method of claim 10, wherein aligning a shroud top with the shroud base comprises:
aligning a first indicium on the shroud top with a second indicium on the shroud base.

13. The method of claim 10, wherein the spike is movable toward the sealed vial through rotation of the shroud top with respect to the shroud base.

14. The method of claim 10, further comprising:
providing a lock on the shroud base to the shroud top that secures the shroud base to the shroud top in a final position after the spike has pierced the sealed vial.

15. The method of claim 10, further comprising:
coupling a spike assembly to the shroud top, the spike assembly comprising the spike and a split valve.

16. The method of claim 10, wherein connecting the shroud top and the shroud base comprises:
applying a force to the shroud top or the shroud base to snap the shroud top and shroud base together.

17. A method for using a protective housing including a shroud top and a shroud base configured to protect a sealed vial by absorbing one or more forces, the method comprising:
removing at least one tamper prevention device from the protective housing;
rotating the shroud top with respect to the shroud base to pierce the sealed vial; and
connecting an injector to the protective housing to provide a path for fluid medicament from the sealed vial after the sealed vial has been pierced.

18. The method of claim 17, further comprising:
coupling a draw-off tube to the injector.

19. The method of claim 18, wherein connecting the injector comprises:
coupling the draw-off tube to connector of the shroud top and the shroud base.

20. The method of claim 17, further comprising:
priming the injector to ready fluid medicament to be injected to a subject.
